(19) United States
(12) Patent Application Publication
(10) Pub. No.: US 2008/0063611 A1
(43) Pub. Date: Mar. 13, 2008

(54) Dentifrice Compositions

(75) Inventors: Robert L. Ibsen, Santa Maria, CA (US); Alan B. Mathews, Santa Maria, CA (US)

Correspondence Address: CROWELL & MORING LLP;INTELLECTUAL PROPERTY GROUP, P.O. BOX 14300, WASHINGTON, DC, 20044-4300, US

(73) Assignee: Den-Mat Holdings LLC, Santa Maria, CA (US)

(21) Appl. No.: 11/851,715

(22) Filed: Sep. 7, 2007

Related U.S. Application Data
(60) Provisional application No. 60/843,699, filed on Sep. 12, 2006.

(51) Int. Cl. A61K 8/21; A61Q 11/00

(52) U.S. Cl. 424/52

(57) ABSTRACT

Stain-removal, desensitizing, anti-tartar and anti-gingivitis dentifrice compositions. The dentifrices comprise a fluoride agent; an abrasive agent; a proteolytic agent; a foaming agent; a sweetening/flavoring agent; an emulsifying agent; either desensitizing, anti-tartar or anti-gingivitis agents or combinations thereof; and a delivery agent. The dentifrices are visually pleasing and impart a soothing sensation during and after brushing.

### FIELD OF THE INVENTION

The present invention relates to dentifrice compositions, methods of manufacturing dentifrice compositions, and methods of using dentifrice compositions. In particular, the present invention relates to dentifrice compositions comprising saponins, Metasomes, and Florasomes.

### BACKGROUND OF THE INVENTION

White teeth have long been considered cosmetically desirable. Unfortunately, teeth almost invariably become discolored. Over time the use of certain foods and tobacco, the process of aging, diseases, trauma, medications, some congenital conditions, and environmental effects cause teeth to become discolored to varying degrees. This inevitable discoloration combined with the desire for whiter teeth have led to a high level of interest in developing compositions and methods for whitening teeth. To this end, people have in the past relied on mechanical cleaning methods, having veneers placed over their teeth or having their teeth chemically bleached in order to achieve a desired level of tooth whiteness.

A tooth is comprised of an inner dentin layer, an outer hard, slightly porous, enamel layer and the acquired pellicle. The natural color of the tooth is opaque to translucent white or off-white. Tooth enamel is predominantly formed from inorganic material, mostly in the form of hydroxyapatite crystals and further contains approximately 5% organic material primarily in the form of collagen. The dentin layer is composed of about 20% protein, including collagen, with the balance consisting of inorganic material, predominantly hydroxyapatite crystals, similar to that found in enamel. The acquired pellicle is a proteinaceous layer on the surface of tooth enamel which reforms rapidly after an intensive tooth cleaning.

Tooth staining can generally be characterized as extrinsic or intrinsic. Staining of the acquired pellicle arises as a result of compounds such as tannins and polyphenolic compounds coming in contact with the tooth when eating, drinking or smoking. These compounds then become trapped in and tightly bound to the proteinaceous layer on the surface of the teeth. This type of staining is extrinsic. Extrinsic staining is typically removed by mechanical methods of tooth cleaning, such as brushing and/or flossing. In contrast, intrinsic staining occurs when staining compounds penetrate the enamel and even the dentin or arise from sources within the tooth. This type of staining cannot typically be addressed via mechanical methods of tooth cleaning. Chemical methods are required to remove this type of staining.

Dentifrices are used to clean, bleach, whiten, and otherwise treat the teeth and gums. Generally, the active ingredients in a dentifrice are contained within a carrier. The carrier is typically a paste or a gel. Each is dispensed through a tubular orifice onto a brushing device or, in some cases, onto a tray, stint or mouth guard. “Gels” are thickened by a gelling agent that hydrogen bonds a dispersion medium to produce a semisolid, transparent, jelly-like, material. In contrast, “pastes” are thickened by the addition of fillers. However, the line between gels and pastes is not always clear. Furthermore, sometimes dentifrices that are opaque and/or contain one or more abrasive fillers are labeled pastes, even if they exhibit gel-like properties. Therefore, in describing the present invention, the term “dentifrice” will be used to clearly indicate that both gels and pastes are embraced.

Saponins are natural detergents found in many plants, especially certain desert plants. The two major commercial sources of saponins are Yucca schidigera, which grows in the arid Mexican desert country of Baja California, and Quillaja saponaria (soapbark tree), found in arid areas of Chile. Saponins have detergent or surfactant properties because they contain both water-soluble and fat-soluble components. They consist of a fat-soluble nucleus, having either a steroid or triterpenoid structure, with one or more side chains of water-soluble carbohydrates (sugars). Yucca saponins have a steroid nucleus (steroidal saponins), while the quillaja saponins have a triterpenoid nucleus. As a consequence of their surface-active properties, saponins are excellent foaming agents, forming very stable foams. Yucca and quillaja extracts are used in beverages, such as root beer and slurpies, to provide the foamy “head.” Because of their surfactant properties, they are used industrially in mining and ore separation, in preparation of emulsions for photographic films, and extensively in cosmetics, such as lipstick and shampoo. Quillaja bark has been used as a shampoo in Chile for hundreds of years, and Native Americans used yucca to make soap. The antifungal and antibacterial properties of saponins are important in cosmetic applications, in addition to their emollient effects.

Quillaja saponins are a mixture of acylated triterpenoid oligoglycosides that are neutrally charged. Unlike its more common foam-forming substitute, sodium lauryl sulfate, it is a non-irritating cleansing agent that is safe for sensitive skin. Additionally, because it is neutrally-charged, it can be used with drug products that would otherwise be rendered inactive by the presence of charged agents, e.g., cetylpyridinium chloride. Specially-purified quillaja extracts are also used to boost oral vaccine's efficacy because they have been shown to increase transdermal transfer. In a dentifrice, increased overall oral health could be achieved by the inclusion of ingredients such as vitamins, amino acids, minerals and the like, that could be introduced via the oral mucosa. The inventors of the instant invention have unexpectedly discovered that saponins are non-irritating and increase the cleansing ability of dentifrices.

U.S. Pat. No. 6,485,711 describes an organic toothpaste comprising saponins as cleaning and foaming agents. The toothpaste described in U.S. Pat. No. 6,485,711 does not, however, contain emollients for the delivery of cosmetic or active ingredients.

Metasomes and Florasomes are examples of emollients that can be used to deliver cosmetic or active ingredients. Metasomes are composed of polyethylene, petrolatum and ethylhexyl palmitate and may also be shaded with appropriate cosmetic or food-grade pigments. U.S. Pat. No. 6,432,421 describes the Metasomes and their production. Florasomes are formed by jojoba esters and are the natural equivalent of Metasomes. U.S. Pat. No. 6,280,746 describes Florasomes and their production. To date, the use of Metasomes and Florasomes in a dentifrice has not been contemplated in the art.

Many dentifrices are known and used in dentistry. Many of the prior known dentifrices do not perform satisfactorily. The instant inventors have surprisingly discovered unique combinations of saponins and emollients which provide improved dentifrices for oral care. This dentifrice is suited for delivering additional oral-care benefits other than stain-removal. For instance, it is possible to incorporate desensitizing agents for individuals with dental hypersensitivity; breath-freshening adjuncts are possible either in the form of zinc or chloride salts, amino acids, or natural ingredients such as grapefruit or grape extracts or added menthol; because the dentifrice does not employ either sodium lauryl sulfate or cocamidopropyl-betaine, it is useful for individuals that suffer from aphthous ulcers (canker sores) so that they can continue to maintain their oral health; tartar control agents, including sodium tripolyphosphate, trisodium phrophosphate and sodium bicarbonate (among others) can be used in another version; silicas with increased abrasivity can also be employed to provide enhanced cleaning capabilities for individuals with high levels of surface staining such as smokers, wine, tea and coffee drinkers, etc.; anti-gingivitis agents such as Triclosan and cetylpyridinium chloride would be advantageously utilized in another embodiment of the invention as the surfactants are nonionic and would not adversely inhibit these agent's effectivities; finally, vitamin and mineral supplements, amino acids, hyaluronic acid and therapeutic agents that can be introduced across the oral mucosa can be added to the dentifrice itself or via the Metasomes or Florasomes.

There remains a need in the art for a stain-removal dentifrice that does not contribute to mucosal irritation and can also employ desensitizing agents, provides long-lasting fresh breath; would be useful for individuals with aphthous ulcers; could be formulated with differing levels of abrasivity to treat individuals with varied levels of extrinsic stains; could incorporate effective levels of both antitartar and anti-gingivitis agents; and could be used to introduce oral and systemic therapeutic agents.

### SUMMARY OF THE INVENTION

One embodiment of the invention is a stain removal dentifrice. The dentifrice includes at least one fluoride agent; at least one abrasive agent; at least one proteolytic agent; at least one foaming agent; at least one sweetening/flavoring agent; at least one emulsifying agent; and at least one emollient agent. The at least one emollient agent includes visible hollow shapes that enclose at least one abrasive polishing agent and at least one breath-freshening flavoring agent.

Other embodiment of the invention includes the elements cited in the stain-removal dentifrice as well as at least one desensitizing agent; and the elements found in the stain-removal dentifrice that would benefit individuals that suffer from aphthous ulcers because the dentifrice employs non-irritating saponins; and the elements disclosed in the stain-removal dentifrice along with anti-tartar and anti-gingivitis agents; and the stain-fighting dentifrice elements with added therapeutic agents.

### DETAILED DESCRIPTION

One embodiment of the present invention is a stain-removal dentifrice composition. The dentifrice comprises a fluoride agent; an abrasive agent; a proteolytic agent; a foaming agent; a sweetening/flavoring agent; an emulsifying agent; and a delivery agent.

Preferably, the fluoride agent comprises sodium fluoride. Preferably, the abrasive agent comprises silica and alumina, each of which is stable in the presence of sodium fluoride. Preferably, the proteolytic agent comprises bromelain. Preferably, the foaming agent comprises saponins. More preferably, the foaming agent comprises yucca saponins or quillaja saponins. More preferably, the foaming agent comprises specially purified quillaja extracts. Preferably, the sweetening agent comprises acesulfame potassium and xylitol. Preferably the emulsifying agent comprises hydrogenated castor oil.

Preferably, the delivery agent comprises soft hollow shapes that are visible to the user and can be loaded with cosmetic and active ingredients, resist dissolution during incorporation and storage in the dentifrice, yet are soft enough to be crushed during brushing. More preferably, the delivery agent comprises Metasomes or Florasomes. Preferably, the Metasomes and Florasomes are loaded with an abrasive polishing agent and a breath-freshening flavoring agent. More preferably, the Metasomes and Florasomes are loaded with a mixture of alumina and menthol. The Metasomes and Florasomes may also be loaded with other flavors, active agents (such as cetylpyridinium chloride, Triclosan, and the like), minerals (such as ortho- or hexametaphosphates for remineralization), vitamins, amino acids, or similar orally-beneficial agents.

The dentifrice of the present invention is characterized by low abrasivity. Low abrasive dentifrices are advantageous because they are not as likely to abrade the soft dentin portion of the tooth and thus will not contribute to gingival recession. This low abrasivity is provided by the combination of the silica and alumina mechanical polishing agents. The inventive dentifrice thus avoids unnecessary removal of tooth structure and because of the use of bromelain, as an effective proteolytic agent, enhances the mechanical stain-removal abilities of silica and alumina. The dentifrice of the present invention also provides a soothing feel to a user's gums and teeth. This feeling is provided in part by the saponins which unlike both sodium lauryl sulfate and cocamidopropyl betaine do not to contribute to mucosal irritation. The hydrating agent, hyaluronic acid, is also included to enhance overall oral health and contribute to oral soothing capabilities. In addition to acting as a sweetening agent, the xylitol used in the present inventive dentifrice provides ancillary benefits including anticariogenic and antibacterial benefits. The dentifrice of the present invention is also characterized by a long shelf life. Unlike dentifrices that do not employ emulsifiers such as hydrogenated castor oil, the inventive dentifrice does not suffer from the problem of synersis that is common with age. The inventive dentifrice is also characterized by a pleasing visual appearance. The appearance is enhanced by the use of Metasomes and Florasomes that are preferably of a color that contrasts with the dentifrice itself and look like crystals. As the Metasomes and Florasomes preferably contain alumina they would be found to be stain-fighting crystals that will gently polish teeth. The Metasomes and Florasomes advantageously resist dissolution during incorporation and storage in the dentifrice, yet are soft enough to be crushed during brushing. By incorporating menthol in the Metasomes and Florasomes, because menthol is solid at room temperature, when brushing the user can experience a tactile “crunching” sensation along with the cooling and long-lasting breath-freshening feeling that menthol affords.

### EXAMPLES

Dentifrice compositions were made from the following components:

### 1—Basic Stain-Fighting Toothpaste (also for individuals suffering from aphthous ulcers):

### Part A

### Water (aqua)

### Bromelain

### Part B

### Sorbitol

### Sodium fluoride

### Acesulfame potassium

### Methylparaben

### Xylitol

### Part C

### Glycerin

### Polyethylene glycol

### Sodium carboxymethylcellulose

### Part D

### Silica, abrasive

### Silica, thickening

### Alumina

### Titanium dioxide

### Part E

### Hydrogenated castor oil

### Flavor (aroma)

### Sodium hyaluronate powder

### DL-a-tocopheryl acetate (Vitamin E)

### Quillaja extract powder

### Part F

### Metasomes® w 20% alumina & 10% menthol

### 2—Stain-Fighting Toothpaste with Desensitizing Agents:

add an effective amount of potassium nitrate to Example 1

### 3—Stain-Fighting Toothpaste with Baking Soda:

add an effective amount of sodium bicarbonate to Example 1

### 4—Stain-Fighting Toothpaste with Tartar Control Agents:

add an effective amount of sodium tripolyphosphate and tetrasodium pyrophosphate to Example 1

### 5—Stain-Fighting Toothpaste with Oxygen:

add calcium peroxide to Example 1

### 6—Stain-Fighting Toothpaste with Desensitizing Agents, Anti-Tartar Agents, Baking Soda and Oxygen:

add an effective amount of potassium nitrate, an effective amount of sodium tripolyphosphate and tetrasodium pyrophosphate, an effective amount of sodium bicarbonate and add calcium peroxide to Example 1

### Note that that similar, more restricted, combinations using the Stain-Fighting Toothpaste basic formulation would also be possible.

### 7—Stain-Fighting Toothpaste with Anti-gingivitis Agents:

add an effective amount of Triclosan or cetylpyridinium chloride to Example 1

### 8—Stain-Fighting Gel (also for individuals suffering from aphthous ulcers):

remove titanium dioxide and alumina from Example 1

### 9—Stain-Fighting Gel with Desensitizing Agents:

add an effective amount of potassium nitrate to Example 8

### 10—Stain-Fighting Gel with Tartar Control Agents:

add an effective amount of sodium tripolyphosphate and tetrasodium pyrophosphate to Example 8

### 11—Stain-Fighting Gel with Oxygen:

add calcium peroxide to Example 8

### 12—Stain-Fighting Gel with Desensitizing Agents, Anti-Tartar Agents, Baking Soda and Oxygen:

add an effective amount of potassium nitrate, an effective amount of sodium tripolyphosphate and tetrasodium pyrophosphate, an effective amount of sodium bicarbonate and add calcium peroxide to Example 8

### Note that that similar, more restricted, combinations using the Stain-Fighting Toothpaste basic formulation would also be possible.

### 13—Stain-Fighting Gel with Anti-gingivitis Agents:

add an effective amount of Triclosan or cetylpyridinium chloride to Example 8
The present invention provides a stain-removal dentifrice that has a low abrasivity and does not contribute to mucosal irritation and does not suffer from synersis. Additionally alternate versions the present invention provide for dentifrices that are aesthetically pleasing, offer desensitizing, antitartar, and antigingivitis benefits, can be made with baking soda and combinations of the foregoing benefits and impart a soothing sensation when used while brushing teeth.

Although particular embodiments of this invention have been disclosed herein for purposes of explanation, further modifications or variations thereof will be apparent to those skilled in the art to which this invention pertains. Further, although certain processes have been described by a number of steps in a particular order, the present invention is not limited to any particular order. Thus, the scope of the present invention is not meant to be limited in any way.

What is claimed is:

1. A stain removal dentifrice composition comprising an opaque paste containing:
at least one fluoride agent;
at least one abrasive agent;
at least one proteolytic agent;
at least one foaming agent;
at least one sweetening and breath-freshening flavoring agent;
at least one emulsifying agent; and
at least one emollient agent,
wherein the at least one emollient agent comprises visible hollow shapes that enclose at least one abrasive agent and at least one flavoring agent.

2. The dentifrice of claim 1 wherein the at least one emollient agent resists dissolution during incorporation and storage in the dentifrice and is soft enough to be crushed during brushing.

3. The dentifrice of claim 1 wherein at least one the emollient agent comprises hollow spheres comprising polyethylene, petrolatum and ethylhexyl palmitate.

4. The dentifrice of claim 1 wherein the at least one emollient agent comprises hollow spheres comprising jojoba esters.

5. The dentifrice of claim 1 wherein the at least one emollient agent further encloses at least one of the elements selected from the group consisting of: active agents, minerals, vitamins, and amino acids.

6. The dentifrice of claim 1 wherein the at least one emollient agent further encloses at least one of the elements selected from the group consisting of: cetylpyridinium chloride and Triclosan.

7. The dentifrice of claim 1 wherein the at least one emollient agent further encloses at least one of the elements selected from the group consisting of: ortho- or hexametaphosphates.

8. A stain-removal and desensitizing dentifrice composition comprising an opaque paste containing the dentifrice of claim 1 with an effective amount of at least one dental desensitizing agent.

9. A stain-removal and desensitizing dentifrice composition comprising an opaque paste containing the dentifrice of claim 1 with an effective amount of anti-tartar agents.

10. The dentifrice of claim 10 where the anti-tartar agents are taken from the group comprised of sodium ripolyphosphate, tetrasodium pyrophosphate, trisodium phosphate, tetrapotassium pyrophosphate and similar phosphate salts.

11. A stain-removal toothpaste with baking soda comprising an opaque paste containing the dentifrice of claim 1 with an effective amount of sodium bicarbonate.

12. A stain-removal and anti-gingivitis dentifrice composition comprising an opaque paste containing the dentifrice of claim 1 with an effective amount of at least one anti-gingivitis agent.

13. The dentifrice of claim 13 where the anti-gingivitis agents are taken from the group comprised of Triclosan and cetylpyridinium chloride.

14. A stain-removal dentifrice with oxygen-releasing ingredients comprising an opaque paste containing the dentifrice of claim 1 with calcium peroxide.

15. A stain-removal dental dentifrice composition comprising a translucent dental gel containing the dentifrice of claim 1.

16. A stain-removal and desensitizing dentifrice composition comprising a translucent dental gel containing the dentifrice of claim 16 with an effective amount of at least one dental desensitizing agent.

17. A stain-removal and desensitizing dentifrice composition comprising a translucent dental gel containing the dentifrice of claim 16 with an effective amount of anti-tartar agents.

18. The dentifrice of claim 18 where the anti-tartar agents are taken from the group comprised of sodium tripolyphosphate, tetrasodium pyrophosphate, trisodium phosphate, tetrapotassium pyrophosphate and similar phosphate salts.

19. A stain-removal and anti-gingivitis dentifrice composition comprising a translucent dental gel containing the dentifrice of claim 16 with an effective amount of at least one anti-gingivitis agent.

20. The dentifrice of claim 20 where the anti-gingivitis agents are taken from the group comprised of Triclosan and cetylpyridimium chloride.

21. A stain-removal dentifrice with oxygen-releasing ingredients comprising a translucent dental gel containing the dentifrice of claim 16 with calcium peroxide.

22. A combination of a stain-removal dentifrices comprising opaque pastes with desensitizing and anti-tartar agents, either with or without baking soda and oxygenating agents.

23. A combination of a stain-removal dentifrices comprising translucent dental gels with desensitizing and anti-tartar agents and oxygenating agents.

24. The translucent gels of claim 24 and opaque pastes with suitable FD&C colorants added for visual effect.

25. Combinations of one or more of a translucent dental gels and one or more of an opaque pastes filled simultaneously into the same tube to impart both visual effect and as a means of delivering combinations of cosmetic and therapeutic benefits.
